(19) United States
(12) Patent Application Publication
(10) Pub. No.: US 2009/0068267 A1
(43) Pub. Date: Mar. 12, 2009

(54) Novel Pharmaceutical Compositions

(75) Inventors: Sergio Lloret Perez, Barcelona (ES); Marina Puigvert Colomer, Barcelona (ES)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Combino Pharm, S.L., Santa Joan Despi (ES)

(21) Appl. No.: 12/205,008

(22) Filed: Sep. 5, 2008

Related U.S. Application Data
(60) Provisional application No. 60/967,612, filed on Sep. 6, 2007.

(51) Int. Cl. A61K 9/28; C07H 17/02; A61K 31/513

(52) U.S. Cl. 424/474; 536/17.4; 514/274

(57) ABSTRACT

The present invention relates to novel lamivudine/cyclodextrin complexes and processes for preparing said complexes. The present invention also relates to novel solid pharmaceutical compositions comprising lamivudine, wherein lamivudine is present in the form of said lamivudine/cyclodextrin complexes. The present invention further relates to processes for preparing said compositions.

### CROSS-REFERENCE TO RELATED APPLICATIONS

This patent application claims the benefit of U.S. Provisional Patent Application Nos. 60/967,612, filed on Sep. 6, 2007, which is incorporated by reference.

### BACKGROUND OF THE INVENTION

Lamivudine is the international common accepted name for (−)-4-amino-1-[(2R,5S)-2-(hydroxymethyl)-1,3-oxathiolan-5-yl]pyrimidin-2(1H)-one, an 1,3-oxathiolane nucleoside analogue. Lamivudine has an empirical formula of C₈H₁₁N₃O₃S, a molecular weight of 229.3 g/mol, and a structure of formula (I):

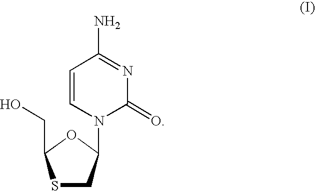

Lamivudine has been described as having antiviral activity, in particular against the human immunodeficiency viruses (HIVs), the causative agents of AIDS.

Polymorphism is defined as the ability of a substance to crystallize in more than one crystal lattice arrangement. Polymorphism can influence many aspects of solid state properties of a drug. Different crystal modifications of a substance can differ considerably from one another in many respects such as their solubility, dissolution rate and finally bioavailability.

It is known that lamivudine exhibits polymorphism. U.S. Pat. No. 5,905,082 (equivalent to EP 0517145B1), which is incorporated herein by reference, discloses that lamivudine can exist in two polymorphic forms, one in the form of needle-shaped crystals, known as polymorphic Form I, and the other in substantially bipyramidyl crystals, known as polymorphic Form II. Lamivudine polymorphic Form I is not suitable for producing solid pharmaceutical compositions because of its physical properties, such as, for example, poor flow characteristics and stability. In addition, lamivudine polymorphic Form I can be unstable as certain pharmaceutical unit operations such as, for example, milling can cause conversion of Form I to Form II, an undesirable characteristic for manufacture of solid pharmaceutical compositions.

Lamivudine polymorphic Form II displays improved flow characteristics and thus, is preferred over polymorphic Form I in the manufacture of solid pharmaceutical compositions.

International Patent Publication No. WO 2007/119248 A1, which is incorporated herein by reference, discloses a novel hemihydrate crystalline form of lamivudine, which is designated therein as Form III.

Crystalline solids normally require a significant amount of energy for dissolution due to their highly organized lattice like structures. For example, the energy required for a drug molecule to escape from a crystal is much higher than the energy required for escaping from either a lower crystalline form, or an amorphous form.

Furthermore, it is well known that active substances in an amorphous form are more soluble and dissolve more rapidly than when in a crystalline form. This improved solubility is manifested as improved bioavailability of the active substance.

However, there are no reports in the literature that lamivudine can exist in an amorphous form, not even in low-crystalline form.

It is also well known that the stability of an active substance depends on the polymorphic form in which it exists and that an amorphous form is less stable than a crystalline form, as the amorphous form is more susceptible to heat, light and moisture. All these factors are of key importance for the stability of pharmaceutical compositions comprising an amorphous substance.

To date, an appropriate and useful pharmaceutical composition comprising lamivudine in non-crystalline forms, even in low-crystalline forms, has not been described.

Accordingly, there exists a need to provide stable pharmaceutical compositions of lamivudine with improved stability comprising lamivudine in non- or low-crystalline forms. Further, there is a need to provide non- or low-crystalline forms of lamivudine or derivatives thereof, which are useful for preparing improved pharmaceutical compositions.

### BRIEF SUMMARY OF THE INVENTION

The present invention relates to novel complexes of lamivudine and cyclodextrin, processes for preparing said novel complexes, and compositions comprising said novel complexes.

In some embodiments, the present invention provides a lamivudine/cyclodextrin complex.

In other embodiments, the present invention provides a lamivudine/β-cyclodextrin complex.

In some embodiments, the present invention provides a process for preparing lamivudine/β-cyclodextrin complexes.

In other embodiments, the present invention provides compositions comprising a lamivudine/cyclodextrin complex.

In other embodiments, the present invention provides a process for preparing compositions comprising a lamivudine/cyclodextrin complex.

### BRIEF DESCRIPTION OF THE DRAWINGS

FIG. 1 illustrates the X-ray powder diffraction pattern (XRD) of the lamivudine/β-cyclodextrin complex as obtained in Example 1.

FIG. 2 illustrates the X-ray powder diffraction pattern (XRD) of a tablet comprising lamivudine in the form of lamivudine/β-cyclodextrin complex as obtained in Example 1.

FIG. 3 illustrates the X-ray powder diffraction pattern (XRD) of crystalline β-cyclodextrin.

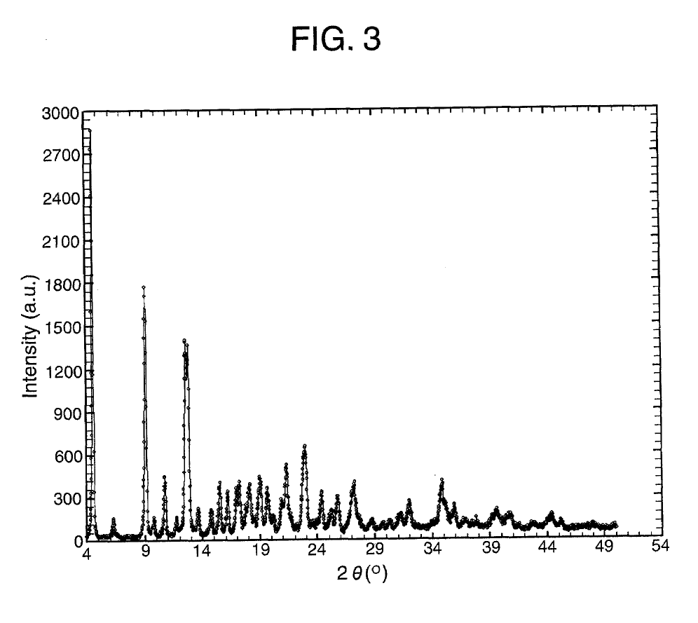

### DETAILED DESCRIPTION OF THE INVENTION

In accordance with the invention, the present invention provides novel lamivudine/cyclodextrin complexes.

Cyclodextrins are crystalline, non-hygroscopic, cyclic oligosaccharides that are useful as pharmaceutically acceptable excipients. Cyclodextrins are used as solubilizing agents since they form inclusion complexes with drug molecules.

Surprisingly, it has been found that when crystalline lamivudine, as obtained by any known method in the art, is combined with a specific amount of a crystalline cyclodextrin, the obtained lamivudine/cyclodextrin complex shows a very low-crystalline form, in which no XRD peak of the lamivudine Forms I, II, or III is observed.

In some embodiments, the present invention provides lamivudine/cyclodextrin complexes which are in a stable low-crystalline state.

In other embodiments, the present invention provides lamivudine/cyclodextrin complexes which are in a non-crystalline state.

Thus, the lamivudine/cyclodextrin complexes of the invention not only display the enhanced stability and solubility profile associated with cyclodextrins, but also display the improved characteristics associated with low-crystalline and/or amorphous forms.

Further, the low-crystalline lamivudine/cyclodextrin complexes of the invention are physically stable, and hence prevent the conversion of lamivudine to crystalline forms, thus avoiding known drawbacks associated with lamivudine polymorphic instability.

Additionally, the lamivudine/cyclodextrin complexes of the invention are physically and chemically stable to certain pharmaceutical unit operations such as compression, thereby making lamivudine/cyclodextrin complexes of the invention amenable to pharmaceutical compounding operations, such as, for example, tabletting.

In some embodiments, the present invention provides a process for preparing a lamivudine/cyclodextrin complex of the invention, wherein the complex is in a stable low-crystalline state or non-crystalline state. Processes in accordance with the invention comprise preparing a mixture of lamivudine and cyclodextrin in water and drying the mixture.

In keeping with processes of the invention, drying of the wet mixtures of lamivudine and cyclodextrin is conducted at a temperature of approximately 50° C. and until constant weight of the mixture is obtained.

In some embodiments, processes of the invention further comprise sieving the dried mixture of lamivudine and cyclodextrin. Typically, sieving of the dried mixture product is carried out through a 1 mm sieve.

In keeping with processes of the invention, the lamivudine used to prepare the lamivudine/cyclodextrin complex can be any lamivudine obtained by any method described in the art.

In keeping with the invention, lamivudine/cyclodextrin complexes of the invention comprise cyclodextrin. Preferably, lamivudine/cyclodextrin complexes of the invention comprise at least about 0.5 molar equivalents of cyclodextrin with respect to the amount of lamivudine. In some preferred embodiments, the present invention provides lamivudine/cyclodextrin complexes comprising about equal molar equivalents of cyclodextrin and lamivudine.

The crystalline cyclodextrin used to prepare the lamivudine/cyclodextrin complex of the invention can be at least one of the group consisting of alpha-, beta-, and gamma-cyclodextrin, derivatives thereof, and mixtures thereof. In preferred embodiments, the crystalline cyclodextrin used to prepare the lamivudine/cyclodextrin complexes of the invention is beta-cyclodextrin, i.e. β-cyclodextrin.

In a preferred embodiment, the lamivudine/cyclodextrin complex of the invention is a lamivudine/β-cyclodextrin complex.

In an embodiment, the invention provides a novel lamivudine/β-cyclodextrin complex, wherein said complex is in a stable low-crystalline state.

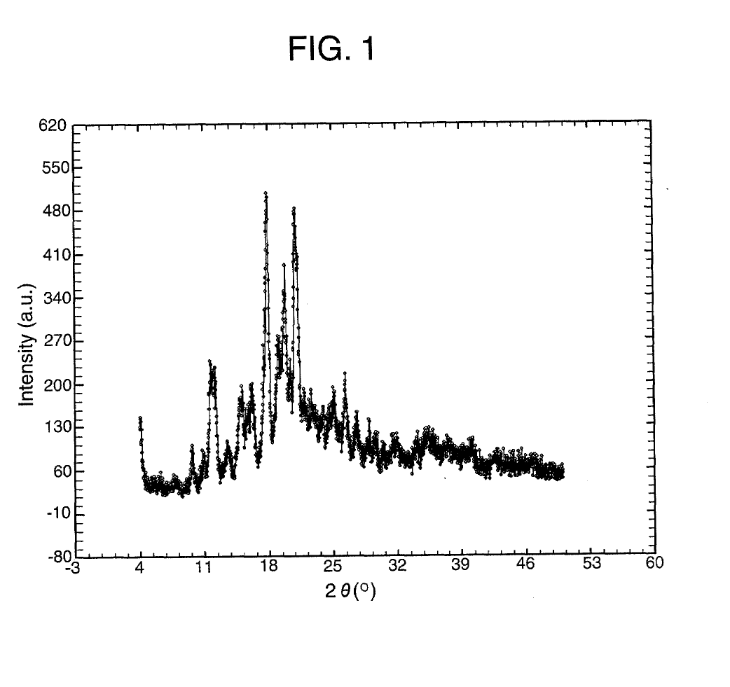

The lamivudine/β-cyclodextrin complex of the present invention has an X-ray diffraction pattern (2θ) as substantially shown in FIG. 1. As depicted in FIG. 1, the low intensity and width of the peaks, and the length of the background suggest that the lamivudine/β-cyclodextrin complex of the present invention has a low crystallinity. Further, no peaks corresponding to lamivudine Forms I, II, or III can be observed in FIG. 1.

In keeping with the invention, a lamivudine/β-cyclodextrin complex of the invention has a stable low-crystalline state for at least 1 year at a temperature of approximately 20-25° C. with approximately 50-60% of relative humidity.

In a preferred embodiment, the present invention provides a lamivudine/β-cyclodextrin complex comprising at least about 0.5 molar equivalents of β-cyclodextrin with respect to the amount of lamivudine.

In other embodiments, the present invention provides lamivudine/β-cyclodextrin complexes comprising about equal molar equivalents of β-cyclodextrin and lamivudine.

Processes in accordance with the invention comprise preparing a mixture of lamivudine and cyclodextrin in water. For example, it has been observed that concentrations of water of less than approximately 15% of weight with respect to the total weight of the lamivudine and β-cyclodextrin do not lead to the complete formation of the lamivudine/β-cyclodextrin complex of the invention. Typically, a mixture of lamivudine and β-cyclodextrin is prepared comprising at least about 15% water by total weight of the mixture, preferably about 15% to about 20% water. In particularly preferred embodiments, mixtures of lamivudine and β-cyclodextrin are prepared comprising about 18% to about 19% water.

In a particularly preferred embodiment, the present invention provides a process for preparing a lamivudine/β-cyclodextrin complex comprising preparing a mixture of lamivudine with β-cyclodextrin in water, drying the mixture, and optionally sieving the dried mixture as described previously.

In some embodiments of the invention, lamivudine/β-cyclodextrin complexes, wherein said complex is in a stable non-crystalline state, are prepared by a process comprising preparing a mixture of lamivudine and β-cyclodextrin in water, drying the mixture, and optionally sieving the dried mixture, wherein said mixture comprises at least about 0.5 molar equivalents of β-cyclodextrin with respect to lamivudine and comprises at least about 15% water by total weight of the mixture before drying.

In other embodiments, the present invention provides a process for preparing a lamivudine/β-cyclodextrin complex, wherein said complex is in a stable low-crystalline state, said process comprising preparing a mixture of lamivudine and β-cyclodextrin in water, drying the mixture, and optionally sieving the dried mixture, wherein said mixture comprises at least about 0.5 molar equivalents of β-cyclodextrin with respect to lamivudine and comprises at least about 15% water by total weight of the mixture before drying.

The lamivudine/β-cyclodextrin complex of the invention is preferably in the form of a solid granule.

In another embodiment, the present invention provides novel solid pharmaceutical compositions comprising lamivudine combined with one or more pharmaceutically acceptable excipients, wherein said lamivudine is present in the form of the lamivudine/cyclodextrin complexes of the invention, which assure the stability and avoid the conversion of lamivudine to crystalline forms, and wherein said solid pharmaceutical composition comprising lamivudine is in a stable low-crystalline form.

In an embodiment, the present invention provides a novel solid pharmaceutical composition comprising lamivudine wherein said lamivudine is present in the form of the lamivudine/β-cyclodextrin complex of the invention, and wherein said solid pharmaceutical composition comprising lamivudine is in a stable low-crystalline form.

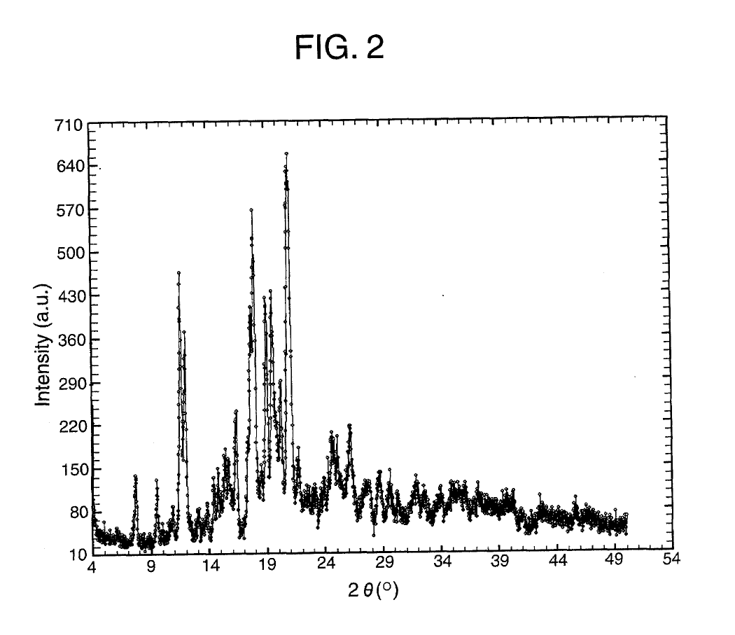

The solid pharmaceutical composition comprising lamivudine wherein said lamivudine is present in the form of the lamivudine/β-cyclodextrin complex of the invention has an X-ray diffraction pattern (2θ) as substantially shown in FIG. 2. As depicted in FIG. 2, the low intensity and width of the peaks, and the length of the background suggest that the solid pharmaceutical composition containing lamivudine of the present invention has a low crystallinity. Further, no peaks corresponding to lamivudine Forms I, II, or III can be observed in FIG. 2.

In keeping with another aspect of the invention, solid pharmaceutical compositions comprising lamivudine wherein said lamivudine is present in the form of a lamivudine/β-cyclodextrin complex of the present invention having a stable low-crystalline state for at least 1 year at a temperature of approximately 20-25° C. with approximately 50-60% of relative humidity.

In other embodiments, the present invention provides solid pharmaceutical compositions which can be in any solid form such as, for example, tablets, orally dispersible pharmaceutical compositions, capsules, pellets, granulate, and the like.

In a particular preferred embodiment, the present invention provides a solid pharmaceutical composition, wherein the composition is a tablet comprising lamivudine wherein the lamivudine is in the form of a lamivudine/β-cyclodextrin complex, wherein said tablet is optionally coated with a coating agent.

In other embodiments, the present invention provides a process for preparing novel solid pharmaceutical compositions comprising lamivudine wherein said lamivudine is present in the form of a lamivudine/cyclodextrin complex, and wherein said solid pharmaceutical composition comprising lamivudine is in a stable low-crystalline form. Processes in accordance with the invention comprise the steps of preparing a mixture of the lamivudine/cyclodextrin complex of the invention with one or more pharmaceutically acceptable excipients, and processing the mixture to obtain a solid pharmaceutical composition in a dosage form selected from the group consisting of a tablet, a capsule, a pellet or a granule.

Suitable pharmaceutically acceptable excipients in accordance with the invention include at least one filler agent and/or at least one disintegrant agent, and/or at least one lubricant agent.

In a preferred embodiment, processing the mixture of lamivudine and cyclodextrin comprises compressing the mixture into a table and optionally, coating the tablet with a coating agent.

In other embodiments, the present invention provides the use of these novel solid pharmaceutical compositions containing lamivudine wherein said lamivudine is present in the form of a lamivudine/β-cyclodextrin complex, and wherein said solid pharmaceutical composition comprising lamivudine is in a stable low-crystalline form according to the invention for the treatment of conditions caused by HIV and hepatitis B infection.

In an embodiment, a pharmaceutical composition of the present invention comprises a lamivudine/β-cyclodextrin complex, a filler agent, a disintegrant agent, and a lubricant agent, wherein said solid pharmaceutical composition comprising lamivudine is in a stable low-crystalline form.

The filler agent of the pharmaceutical composition of the present invention preferably is microcrystalline cellulose.

The disintegrant agent of the pharmaceutical composition of the present invention preferably is sodium croscarmellose.

The lubricant agent of the pharmaceutical composition of the present invention preferably is magnesium stearate.

Optionally, the pharmaceutical composition of the present invention comprises a coated layer.

The following examples further illustrate the invention but, of course, should not be construed as in any way limiting its scope.

### EXAMPLE 1

### Preparation of a Lamivudine Tablet Wherein the Lamivudine is Present in the Form of a Lamivudine/β-Cyclodextrin Complex

This example demonstrates a tablet composition comprising lamivudine as an active pharmaceutical ingredient and one or more pharmaceutically acceptable excipients, wherein said lamivudine is present in the form of the lamivudine/β-cyclodextrin complex, in accordance with an embodiment of the invention. This example further demonstrates a process for preparing a solid pharmaceutical composition in accordance with an embodiment of the invention.

The tablet was prepared using the materials listed in Table 1.

TABLE 1

| Material | Amount (mg/tablet) |
|---|---|
| lamivudine/β-cyclodextrin complex | 1,042.6 |
| (300 mg/742.6 mg) | |
| microcrystalline cellulose | 87.8 |
| sodium croscarmellose | 60.0 |
| magnesium stearate | 9.6 |
| water* | — |
| CORE | 1,200.0 |
| COATING | 30.0 |

*Purified water used during the process is removed and is not present in the final tablet.

The tablet was manufactured using the following procedure comprising the following steps: i) lamivudine and β-cyclodextrin were weighted, transferred into a blender, and mixed to form a homogeneous powder mixture; ii) water was added and mixed to form a homogeneous powder mixture; iii) the mixture of step ii) was dried at 50° C. until constant weight (ca. during 1 hour); iv) the product was passed through a 1 mm sieve, to obtain the lamivudine/β-cyclodextrin complex in the form of granules; v) sodium croscarmellose and microcrystalline cellulose were weighted, passed through a 0.8 mm sieve, and mixed with the granules of step iv); vi) the resultant mixture was compressed into tablets of appropriate weight and hardness to obtain a lamivudine tablet wherein the lamivudine is present in the form of a lamivudine/β-cyclodextrin complex; and vii) optionally, the tablets can be coated.

The granules obtained in step iv), that is, lamivudine/β-cyclodextrin complex, had a XRD as shown in FIG. 1.

The tablets obtained in step vi), that is, a tablet wherein the lamivudine is present in the form of a lamivudine/β-cyclodextrin complex, had a XRD as shown in FIG. 2.

### EXAMPLE 2

### Crystal Stability Studies

This example demonstrates the stability of granules and tablets comprising lamivudine/β-cyclodextrin complex in accordance with the invention.

A sample of the granules of step iv) and of the tablets of step vi) obtained in Example 1 were left for one year at a temperature of approximately 20-25° C. with approximately 50-60% of relative humidity. After one year, the samples were analyzed by X-ray diffraction.

The granules of step iv), that is, lamivudine/β-cyclodextrin complex, had a XRD substantially identical to FIG. 1.

The tablets obtained in step vi), that is, a tablet wherein the lamivudine is present in the form of a lamivudine/β-cyclodextrin complex, had a XRD substantially identical to FIG. 2.

### EXAMPLE 3

### Dissolution of Lamivudine Tablets of Example 1

This example demonstrates the dissolution properties of a tablet prepared in accordance with the invention.

The tablets of Example 1 and commercially available lamivudine tablets (i.e., Epivir® 300 mg) were tested for in vitro drug release in 900 mL of 0.1N HCl using a USP-2 apparatus speed operating at 75 rpm. In both cases the values for the tablets of Example 1 and the values for the commercially available Lamivudine tablets (i.e., Epivir® 300 mg) were greater than 85%. The Biopharmaceutical Classification System (BCS) specifies that dissolution values greater than 85% in 0.1N HCl in 15 minutes can ensure that the bioavailability of the drug is not limited by dissolution. In these cases, the rate limiting step for drug absortion is gastric emptying.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A lamivudine/cyclodextrin complex.

2. The lamivudine/cyclodextrin complex of claim 1, wherein said complex is in a stable low-crystalline state.

3. A process for preparing the lamivudine/cyclodextrin complex of claim 1 or 2, said process comprising:
i) preparing a mixture of lamivudine and cyclodextrin in water; and
ii) drying the mixture of step i); and
iii) optionally, sieving the dried mixture of step ii).

4. The process of claim 3, wherein the cyclodextrin comprises at least one cyclodextrin selected from the group consisting of α-, β-, and γ-cyclodextrin, derivatives thereof, and mixtures thereof.

5. A lamivudine/cyclodextrin complex, wherein said complex is a lamivudine/β-cyclodextrin complex.

6. The lamivudine/β-cyclodextrin complex of claim 5, wherein said complex is in a stable low-crystalline state.

7. The lamivudine/β-cyclodextrin complex of claim 6, wherein said complex is in a non-crystalline state.

8. The lamivudine/β-cyclodextrin complex of any one of claims 5-7, wherein said complex comprises at least about 0.5 molar equivalents of β-cyclodextrin with respect to the amount of lamivudine.

9. The lamivudine/β-cyclodextrin complex of claim 8, wherein said complex comprises about equal molar equivalents of β-cyclodextrin and lamivudine.

10. A process for preparing the lamivudine/β-cyclodextin complex of any one of claims 5-7, said process comprising:
i) preparing a mixture of lamivudine with β-cyclodextrin in water; and
ii) drying the mixture of step i); and
iii) optionally, sieving the dried mixture of step ii);
wherein, said mixture of step i) comprises at least about 0.5 molar equivalents of β-cyclodextrin with respect to lamivudine and comprises at least about 15% water by total weight.

11. The process of claim 10, wherein the mixture of step i) comprises about equal molar equivalents of β-cyclodextrin and lamivudine.

12. The process of claim 10, wherein the mixture of step i) comprises about 15% to about 20% water.

13. The process of claim 10, wherein the mixture of step i) comprises about 18% to about 19% water.

14. A solid pharmaceutical composition comprising lamivudine and one or more pharmaceutically acceptable excipients, wherein said lamivudine is present in the form of the lamivudine/cyclodextrin complex of claim 1 or 2.

15. A solid pharmaceutical composition comprising lamivudine and one or more pharmaceutically acceptable excipients, wherein said lamivudine is present in the form of the lamivudine/β-cyclodextrin complex of any one of claims 5-7.

16. The composition of claim 15, wherein said composition is in a stable low-crystalline state.

17. The composition of claim 15, wherein said composition is in the form of a tablet, an orally dispersible pharmaceutical composition, a capsule, a pellet, or a granule.

18. The composition of claim 15, wherein said composition is in the form of a tablet.

19. A process for preparing the solid pharmaceutical composition of claim 17, said process comprising:
i) preparing a mixture of a lamivudine/β-cyclodextrin complex with one or more pharmaceutically acceptable excipients; and
ii) processing the mixture of step i) to obtain a solid pharmaceutical composition in a dosage form selected from the group consisting of a tablet, an orally dispersible pharmaceutical composition, a capsule, a pellet, or a granule.

20. The process of claim 19, step ii) comprises compressing the mixture of step i) into a tablet, and optionally, coating said tablet with a coating agent.

21. A tablet formulation comprising lamivudine, wherein the lamivudine is in the form of a lamivudine/β-cyclodextrin complex, and wherein said tablet is optionally coated with a coating agent.

22. A lamivudine/cyclodextrin complex of claim 1, wherein said complex has a XRD pattern in which no XRD peak corresponding to lamivudine Form I, II, or III is observed.
